(19) United States
(12) Patent Application Publication
(10) Pub. No.: US 2013/0231508 A1
(43) Pub. Date: Sep. 5, 2013

(54) BRANCHED SECONDARY ALCOHOL ALKOXYLATE SURFACTANTS AND PROCESS TO MAKE THEM

(71) Applicants: Shawn J. Maynard, Angleton, TX (US); Wanglin Yu, Pearland, TX (US); Daniel A. Aguilar, Lake Jackson, TX (US)

(72) Inventors: Shawn J. Maynard, Angleton, TX (US); Wanglin Yu, Pearland, TX (US); Daniel A. Aguilar, Lake Jackson, TX (US)

(73) Assignee: DOW GLOBAL TECHNOLOGIES LLC, Midland, MI (US)

(21) Appl. No.: 13/879,691

(22) PCT Filed: Nov. 3, 2011
(86) PCT No.: PCT/US11/59058

§ 371 Date: Apr. 16, 2013

Related U.S. Application Data
(60) Provisional application No. 61/416,462, filed on Nov. 23, 2010.

(51) Int. Cl. C07C 43/11; C07C 41/03

(52) U.S. Cl. CPC C07C 43/11; C07C 41/03
USPC 568/618; 568/622; 568/678

(57) ABSTRACT

Provided are alkoxylates of the formula I: Also provided is a process for making alkoxylates of formula I. The process provides alkoxylates that exhibit narrow molecular weight distribution and low amounts of residual unreacted alcohol. The alkoxylates have utility in a variety of applications, such as use as surfactants.

### CROSS-REFERENCE TO RELATED APPLICATIONS

This application claims priority from provisional application Ser. No. 61/416,462, filed Nov. 23, 2010, which is incorporated herein by reference in its entirety.

### FIELD OF THE INVENTION

The invention relates to alkoxylate compositions and to processes for making and using them. The alkoxylate compositions exhibit favorable properties such as narrow molecular weight distribution and low content of residual alcohol.

### BACKGROUND OF THE INVENTION

Alcohol ethoxylates are an industrially important class of materials that find use in a wide variety of applications, for instance, as surfactants and detergents. Primary alcohol ethoxylates are conventionally prepared by base catalyzed ethoxylation of a primary alcohol. The simplicity of the manufacturing process and its ability to provide quality products (e.g., narrow molecular weight distribution and/or low residual alcohol content) has resulted in a wide variety of these types of materials being prepared.

In contrast to primary alcohols, highly branched secondary alcohols are considerably less reactive and therefore much more difficult to ethoxylate by the base catalyzed process. As a result, alternative procedures for manufacture of highly branched secondary alcohol ethoxylates have been developed.

A commonly used alternative is based on a two-step process. In step one, an alcohol or alcohol mixture is treated with ethylene oxide (EO) in the presence of a Lewis acid catalyst, BF₃ is commonly used, to add a small amount of EO to the alcohol. The low EO adduct is purified by thorough washing to remove the catalyst and by-products and then subjected to distillation to separate the desired product from unreacted alcohols and lower adducts. The purified low EO product (average 2-4 mole EO) is carried to step two in which a base-catalyzed conventional alkoxylation is performed to produce the final surfactant products.

The two-step process has a number of disadvantages. For instance, the product from the first step generally contains considerable amount of byproduct 1,4-dioxane that needs to be removed. In addition, the ethoxylate products typically exhibit an unfavorably broad molecular weight distribution and a large amount of unreacted alcohol starting material. As a result, if final materials of acceptable quality are to be prepared, isolation and purification of intermediates is needed. Such isolation and purification, and the additional second alkoxylation process, however, significantly increase the cost of the process and result in the generation of large amounts of waste.

New highly branched secondary alcohol alkoxylates that exhibit narrow molecular weight distributions and low content of residual alcohols, as well as low-cost and low waste-generating processes for making them, would be a significant advance in the art.

### BRIEF SUMMARY OF THE INVENTION

In one aspect, the invention provides an alkoxylate composition that exhibits narrow molecular weight distribution. In some embodiments, the alkoxylate composition may also contain low content of residual unreacted alcohol. The composition comprises one or more alkoxylates of formula I:

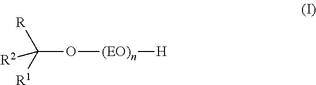

wherein EO, n, R, R¹ and R² are as defined below.

In another aspect, the invention provides a process for making an alkoxylate of formula I. The process comprises: reacting under alkoxylation conditions a secondary alcohol having 7 to 16 carbon atoms and a branching degree of 3 or more with ethylene oxide. The alkoxylation is conducted in the presence of a double metal cyanide catalyst.

### DESCRIPTION OF THE FIGURES

FIG. 1 shows GPC chromatograms from ethoxylation of 2,6,8-trimethylnonan-4-ol (TMN) with 2.0 mole of ethylene oxide (EO) using KOH compared with using double metal cyanide (DMC) catalysts.

FIG. 2 shows GPC chromatograms from ethoxylation of 2,6,8-trimethylnonan-4-ol (TMN) with 6.6 mole of ethylene oxide (EO) using KOH compared with using double metal cyanide (DMC) catalysts.

FIG. 3 shows GPC chromatograms from ethoxylation of 2,6,8-trimethylnonan-4-ol (TMN) with 9.5 mole of ethylene oxide (EO) using KOH compared with using double metal cyanide (DMC) catalysts.

### DETAILED DESCRIPTION OF THE INVENTION

As noted above, in a first aspect the invention provides a composition comprising one or more alkoxylates of the formula I:

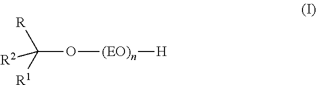

wherein EO is ethyleneoxy; n is 1-40; R and R¹ are independently C₁-C₁₄ alkyl; and R² is H or C₁-C₁₃ alkyl, wherein the group formed by R, R¹, R² and the carbon to which they are attached contains 7 to 16 carbon atoms and has a branching degree of at least 3.

Alkoxylates of formula I prepared according to the processes described herein have been surprisingly discovered to exhibit a narrow molecular weight distribution, represented by the materials' polydispersity index (weight average molecular weight/number average molecular weight (Mw/Mn) as determined by gel permeation chromatography). A narrow molecular weight distribution generally results in better surfactant performance. In some embodiments, the polydispersity index (PDI) of the alkoxylates is 2.0 or less, alternatively 1.75 or less, alternatively 1.5 or less, alternatively 1.2 or less, or alternatively 1.15 or less.

In addition to exhibiting low PDI, in some embodiments, alkoxylates of formula I may also be prepared as described herein to contain surprisingly low levels of residual unreacted alcohols. In contrast, alkoxylates containing the same or similar number of alkylene oxide repeat units prepared by traditional potassium hydroxide catalyzed reaction contain considerably greater amounts of residual alcohols (see the Examples). The advantages of having low levels of alcohols include enhanced surface activity, low odor, and improved clarity of aqueous formulations. In some embodiments, the compositions of the invention contain 10 weight percent or less, alternatively 5 weight percent or less, alternatively 3 weight percent or less, alternatively 2 weight percent or less, alternatively 1 weight percent or less, or alternatively 0.5 weight percent or less of residual alcohols.

Formula I includes variable “n” that describes the molar amount of charged ethylene oxide used in making the compound. In some embodiments, n is at least about 2, alternatively at least about 3, alternatively at least about 4, alternatively at least about 5, alternatively at least about 6, alternatively at least about 7, or alternatively at least about 8. In some embodiments, n is about 30 or less, alternatively about 20 or less, alternatively about 15 or less, or alternatively about 12 or less. In some embodiments, n falls in the range of from about 2 to about 15, alternatively about 4 to about 15, or alternatively about 8 to about 15. In some embodiments, n is about 8. In some embodiments, n is about 11.

In the formula I alkoxylates, R, R¹, R² and the carbon to which they are attached form a group that is the organic residue of the highly branched secondary alcohol used to make the alkoxylate. In general, the group contains between 7 and 16 carbon atoms. In some embodiments, the group contains between 9 and 12 carbon atoms. The group also has a branching degree of 3 or more. In some embodiments of the invention, the branching degree is 4 or more. The term “branching degree” as used herein means the total number of methyl (—CH₃) groups minus 1. For instance, if there are four methyl groups, then the branching degree is 3.

In some embodiments of the invention, R is C₃-C₁₂ alkyl, alternatively C₃-C₈ alkyl, or alternatively C₄-C₆ alkyl. In some embodiments, R contains at least 2 methyl groups.

In some embodiments of the invention, R¹ is C₃-C₁₂ alkyl, alternatively C₄-C₁₀ alkyl, or alternatively C₆-C₈ alkyl. In some embodiments, R¹ contains at least 2 methyl groups.

In some embodiments of the invention, R² is C₁-C₃ alkyl. In some embodiments, R² is H.

In some embodiments of the invention, the alkoxylate is of the formula II:

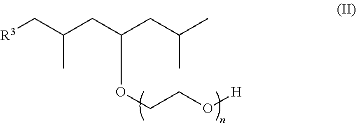

wherein R³ is H or iso-propyl and n is as defined above.

In some embodiments of the invention, the alkoxylate is of the formula:

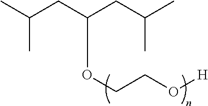

wherein n is as defined above.

In some embodiments, the alkoxylate is of the formula:

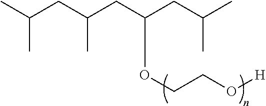

wherein n is as defined above.

In another aspect, the invention provides a process for making the alkoxylates of formula I. According to the process, a highly branched secondary alcohol is reacted with ethylene oxide, under alkoxylation conditions in the presence of a catalyst. The catalyst used for the alkoxylations is a double metal cyanide compound.

The highly branched secondary alcohol is a compound containing 7 to 16 carbon atoms, a branching degree of 3 or more, and one hydroxy group. In some embodiments, the compound contains between 9 and 12 carbon atoms. In some embodiments, the branching degree is 4 or more. Examples of suitable secondary alcohols include 2,6,8-trimethyl-4-nonanol, and 2,6-dimethyl heptan-4-ol.

Prior to the alkoxylation reaction, it may be advantageous to dry the starting alcohol in order to reduce its water content. Various techniques may be used, including for instance application of reduced pressure, elevated temperature, nitrogen purge, or a combination of these. The water content may be reduced to, for example, 300 ppm or less, alternatively 200 ppm or less, or alternatively 100 ppm or less, or alternatively 50 ppm or less, or alternatively 25 ppm or less.

The ethylene oxide is reacted with the alcohol under alkoxylation conditions. In a non-limiting embodiment illustrative of suitable alkoxylation conditions, this reaction may be carried out at an elevated temperature or temperatures ranging from about 80° C. to about 180° C. In other non-limiting embodiments, the temperature may range from about 100° C. to about 160° C. Pressures from about 14 psia to about 60 psia may, in certain non-limiting embodiments, be particularly efficacious, but other pressures may also be effectively employed. Those skilled in the art will be able to determine appropriate conditions with, at most, routine experimentation.

The alkoxylation reaction is conducted in the presence of an effective amount of a double metal cyanide compound as catalyst. The amount of the catalyst may, in some embodiments, range from about 1 ppm to about 1000 ppm by weight, based on the total charge of alcohol and oxides. In some embodiments, the amount may range from about 10 ppm to about 300 ppm. Suitable double metal cyanide catalysts include those described in U.S. Pat. No. 6,429,342, which is incorporated herein by reference. By way of example, Zn₃[Co(CN)₆]₂ may be used as the catalyst.

In a typical process illustrative of the invention, the catalyst may be dissolved or dispersed in the dried alcohol or, alternatively, the two may be mixed first and then the alcohol dried, e.g., using the techniques discussed above, to reduce the residual water content. The ethylene oxide may then be continuously added and the reaction continued until a desired level of alkoxylation has occurred. In some embodiments, the ethylene oxide may instead be added in a batch manner, such as through two, three, or four charges throughout the reaction process. The reaction may be subjected to digestion periods (e.g., about 1-10 hours at about 100 to 160° C.) between ethylene oxide additions and/or after the final ethylene oxide addition.

Following the alkoxylation reaction, the product may be discharged from the reactor directly to be packaged without removal of the catalyst. If desired, the product may be filtered prior to packaging or use, or treated by different means to remove or recover the catalyst, such as taught in U.S. Pat. Nos. 4,355,188; 4,721,818; 4,877,906; 5,010,047; 5,099,075; 5,416,241, each of which is incorporated herein by reference.

The product may also be subjected to additional purification steps. For instance, in some embodiments, the level of residual alcohol may be further reduced by heating the crude ethoxylated product at elevated temperature, such as 120° C. or greater, alternatively 150° C. or greater. In addition, in some embodiments, a vacuum may be applied, e.g., 250 Torr or less, or 200 Torr or less, or 150 Torr or less, such that the boiling point of any residual alcohol is exceeded. An inert gas, such as nitrogen, may be flowed over (head-space sparge) or through (sub-surface sparge) the product to further facilitate removal of the alcohol. Combinations of the foregoing techniques may be applied.

The final formula I alkoxylate of the invention may be used in formulations and compositions in any desired amount. By way of example, when used as a surfactant, typical amounts in many conventional applications may range from about 0.05 to about 90 weight percent, more frequently from about 0.1 to about 30 weight percent, and in some uses from about 0.5 to about 20 weight percent, based on the total formulation. Those skilled in the art will be able to determine usage amounts via a combination of general knowledge of the applicable field as well as routine experimentation where needed.

Applications of the alkoxylates of the invention may include a wide variety of formulations and products. These include, but are not limited to, as surfactant, or wetting, emulsifying, solubilizing, dispersing, demulsifying, cleaning, foam controlling agents, or adjuvant, or combination of these functions in cleaners, detergents, hard surface cleaning formulations, polyurethanes, epoxies, emulsion polymerization, thermoplastics, metal products, agricultural products including herbicides and pesticides, oilfield products and processes, pulp and paper products, textiles, water treatment products, flooring products, inks, colorants, pharmaceuticals, cleaning products, personal care products, and lubricants. As an example of the dispersing application, the alkoxylates of the invention may be used as dispersing agents for fluororesins.

The following examples are illustrative of the invention but are not intended to limit its scope. Unless otherwise indicated, the ratios, percentages, parts, and the like used herein are by weight.

### EXAMPLES

### Raw Materials

2,6,8-Trimethylnonan-4-ol (TMN) and 2,6-dimethyl heptan-4-ol (diisobutyl carbinol or DIBC) are supplied by The Dow Chemical Company.

Double metal cyanide (DMC) catalyst is supplied by Bayer.

Ethylene Oxide (EO) is supplied by The Dow Chemical Company.

### Manufacturing Equipment

DMC catalyzed alkoxylate samples are prepared using a semi-batch process in a 9 liter, stirred, baffled, and jacketed reactor.

### Property Test Methods

Conventional GPC is used for general molecular weight analysis. Reported results are relative to linear polyethylene glycol standards. Polymer Laboratories PEG-10 Polyethylene glycol standards are used with 3^{rd} order fitting. Molecular weight is measured with an Agilent 1100 system equipped with a Polymer Labs Mixed E column coupled to a Differential Refractive Index detector operated at 40° C. The chromatographic mobile phase is tetrahydrofuran (THF). Each sample (100 ul, 25 mg/mL) is dissolved in THF, injected twice, and eluted at 1.0 mL/min.

% OH and hydroxyl equivalent molecular weight (HEMW) are determined on alkoxylate samples by titration according to ASTM D4274 (Test method B). The HEMW is calculated using Eq. 1.

$$H E M W = \frac{1700 N}{\% OH} \qquad Eq . 1$$

where N is the functionality of the sample (1 in the case of the secondary alcohol ethoxylate monols used in the present study).

The amount of unreacted alcohol in alkoxylate samples is determined by gas chromatography, using the response of an internal standard, 1-nonanol. Approximately 0.05 g of ethoxylate sample and 0.03 to 0.1 g of internal standard stock solution (n-nonanol in hexane, 9.9% (w/w)) are weighed (nearest 0.1 mg) into an auto-sampler vial. The samples are derivatized for 15 minutes at 60° C. using 1 mL of Regisil (99% BSTFA and 1% TMCS)) to increase the volatility of the high molecular weight components. The samples are further diluted, as necessary, in hexane and tetrahydrofuran. Derivatized samples are evaluated with an Agilent model 6890 instrument equipped with an HP-7673 auto sampler, an on-column inlet, and a flame ionization detector. The alcohol concentration data reported are from single injections.

### Example 1

### Ethoxylation of TMN Alcohol

The ethoxylates of TMN alcohol is prepared by reaction between EO and TMN in the presence of the DMC catalyst. DMC catalyst (0.15 g) is slurried into 1,193 g of dried (90° C., with nitrogen sweep, until water is less than 200 ppm (23 ppm)) starter alcohol (TMN), activated (200 g EO, 130° C., under 20 psia nitrogen), and then 626 g EO is added (826 g total) continuously (5 g/min) with stirring resulting in the alkoxylate product after 81 min digestion period (130° C.). An intermediate sample (1-A in Table 1, 120 g) is removed. The reaction product measures a hydroxyl content of 5.37% OH and hydroxyl equivalent molecular weight (HEMW) of 317, corresponding to the 2.9 EO/TMN molar ratio alkoxylate. Subsequently, a second ethylene oxide (1,268 g EO, 2,094 g EO total) feed (5 g/min) and digestion period (67 minute, 130° C.) are applied. An intermediate sample (1-B, 123 g) is removed. The reaction product measures a hydroxyl content of 3.33% OH and HEMW of 511, corresponding to the 7.3 EO/TMN molar ratio alkoxylate. Subsequently, a third ethylene oxide (879 g EO, 2,976 g EO total) feed (5 g/min) and digestion period (7 hour, 130° C.) are applied. The reaction product (1-C) measures a hydroxyl content of 2.56% OH and HEMW of 663, corresponding to the 10.8 EO/TMN molar ratio alkoxylate. As listed in Table 1, the TMN/2.9EO sample contains 18.6 wt % of unreacted TMN alcohol residue and has PDI of 1.24. The TMN/7.3EO sample contains 2.8 wt % unreacted TMN alcohol residue and has PDI of 1.13. The TMN/10.8EO sample contains 2.3 wt % unreacted TMN alcohol residue and has PDI of 1.24.

Following the same procedure, other TMN/EO products are prepared and listed in Table 1 (Examples 2 to 8).

TABLE 1
Property Results for DMC Catalyzed TMN Alcohol Alkoxylates

| Ex/ | | | | | | | | | Residual |
|---|---|---|---|---|---|---|---|---|---|
| Sample | De- | % | | Water^{a} | | | | | TMN |
| No. | scription | OH | HEMW | (ppm) | Mₙ | M_{w} | M_{z} | PDI | (wt %) |
| 1-A | TMN/ | 5.37 | 317 | 23 | 361 | 446 | 550 | 1.24 | 18.6 |
| | 2.9 EO | | | | | | | | |
| 1-B | TMN/ | 3.33 | 511 | — | 550 | 624 | 696 | 1.13 | 2.8 |
| | 7.3 EO | | | | | | | | |
| 1-C | TMN/ | 2.56 | 663 | — | 514 | 636 | 755 | 1.24 | 2.3 |
| | 10.8 EO | | | | | | | | |
| 2 | TMN/ | 5.81 | 292 | 189 | 379 | 496 | 763 | 1.31 | 29.2 |
| | 2.3 EO | | | | | | | | |
| 3 | TMN/ | 5.77 | 295 | 63 | 370 | 505 | 950 | 1.37 | 28.6 |
| | 2.4 EO | | | | | | | | |
| 4-A | TMN/ | 5.78 | 290 | 25 | 389 | 477 | 607 | 1.23 | 28.9 |
| | 2.4 EO | | | | | | | | |
| 4-B | TMN/ | 3.27 | 520 | — | 622 | 726 | 847 | 1.17 | 4.9 |
| | 7.5 EO | | | | | | | | |
| 5-A | TMN/ | 2.44 | 698 | 129 | 718 | 782 | 834 | 1.09 | 2.7 |
| | 11.5 EO | | | | | | | | |
| 5-B | TMN/ | 2.13 | 799 | — | 744 | 831 | 886 | 1.12 | 1.5 |
| | 13.8 EO | | | | | | | | |
| 5-C | TMN/ | 1.95 | 873 | — | 863 | 917 | 965 | 1.06 | 0.9 |
| | 15.5 EO | | | | | | | | |
| 6-A | TMN/ | 2.75 | 617 | 271 | 620 | 686 | 734 | 1.11 | 0.9 |
| | 9.7 EO | | | | | | | | |
| 6-B | TMN/ | 2.63 | 647 | — | 605 | 695 | 749 | 1.15 | 0.8 |
| | 10.4 EO | | | | | | | | |
| 6-C | TMN/ | 2.51 | 677 | — | 608 | 693 | 761 | 1.14 | 1.2 |
| | 11.1 EO | | | | | | | | |
| 7 | TMN/ | 3.54 | 480 | 271 | 490 | 556 | 611 | 1.14 | 5.8 |
| | 6.6 EO | | | | | | | | |
| 8 | TMN/ | 3.38 | 503 | 192 | 540 | 606 | 666 | 1.12 | 3.8 |
| | 7.1 EO | | | | | | | | |

^{a}Water content in alcohol initiator

### Example 9

### Direct Ethoxylation of DIBC Alcohol Catalyzed by DMC

The ethoxylates of DIBC alcohol is prepared by reaction between EO and DIBC in the presence of the DMC catalyst. DMC catalyst (0.16 g) is slurried in 1,535 g of dry (90° C., with nitrogen sweep, until water is less than 200 ppm (23 ppm)) starter alcohol (DIBC), activated (215 g EO, 130° C., under 20 psia nitrogen), and then 1,320 g EO is added (1,535 g total) continuously (5 g/min) with stirring resulting in the alkoxylate product after 75 min digestion period (130° C.). The reaction product (9, in Table 2) is removed and measures a hydroxyl content of 2.90% OH and a HEMW of 587, corresponding to the 10.0 EO/DIBC molar ratio alkoxylate. As listed in Table 2, the DIBC/10.0EO sample contains 0.2 wt % of unreacted DIBC alcohol residue and has PDI of 1.04.

Following the same procedure, other DIBC/EO products are prepared as listed in Table 2 (Examples 9-11).

TABLE 2
Property Results for DMC Catalyzed DIBC Alkoxylates

| | | | | | | | | | Un- |
|---|---|---|---|---|---|---|---|---|---|
| Ex/ | | | | | | | | | reacted |
| Sample | De- | % | | Water^{a} | | | | | DIBC |
| No. | scription | OH | HEMW | (ppm) | Mₙ | M_{w} | M_{z} | PDI | (wt %) |
| 9 | DIBC/ | 2.90 | 587 | 23 | 631 | 658 | 684 | 1.04 | 0.2 |
| | 10 EO | | | | | | | | |
| 10-A | DIBC/ | 6.78 | 251 | 147 | 275 | 316 | 356 | 1.15 | 6.8 |
| | 2.4 EO | | | | | | | | |
| 10-B | DIBC/ | 4.71 | 361 | — | 381 | 424 | 460 | 1.11 | 2.2 |
| | 4.9 EO | | | | | | | | |
| 10-C | DIBC/ | 4.49 | 379 | — | 406 | 465 | 553 | 1.15 | 1.8 |
| | 5.3 EO | | | | | | | | |
| 11-A | DIBC/ | 3.74 | 454 | 147 | 476 | 516 | 550 | 1.08 | 0.6 |
| | 7.1 EO | | | | | | | | |
| 11-B | DIBC/ | 3.00 | 566 | — | 571 | 605 | 634 | 1.06 | 0.1 |
| | 9.6 EO | | | | | | | | |

^{a}Water content in alcohol initiator

### Comparative Example 12

### TMN/EO Alkoxylates Prepared with KOH

The alkoxylate product is prepared by reaction between EO and TMN in the presence of the KOH catalyst. KOH catalyst (5.80 g, 45% aqueous solution, 2.55 g contained KOH) is dissolved in 999 g of TMN alcohol, stripped (90° C., under vacuum, with nitrogen sweep, until water is less than 300 ppm (215 ppm)), activated (200 g EO, 130° C., under 20 psia nitrogen), and then 1,425 g EO added (1,625 g total) continuously (5 g/min) with stirring resulting in the alkoxylate product after 120 min digestion period (130° C.). An intermediate sample (12-A, 122 g) is removed. The reaction product measures a hydroxyl content of 3.5% OH and HEMW of 486, corresponding to the 6.7 EO/TMN molar ratio alkoxylate. Subsequently, a second ethylene oxide (552 g EO, 2,177 g EO total) feed (5 g/min) and digestion period (122 minute, 130° C.) are applied. An intermediate sample (12-B, 153 g) is removed. The reaction product measures a hydroxyl content of 2.8% OH and HEMW of 601, corresponding to the 9.4 EO/TMN molar ratio alkoxylate. Subsequently, a third ethylene oxide (607 g EO, 2,784 g EO total) feed (5 g/min) and digestion period (112 minute, 130° C.) are applied. The reaction product (12-C, 3,241 g) measures a hydroxyl content of 2.2% OH and HEMW of 762, corresponding to the 13.0 EO/TMN molar ratio alkoxylate. As listed in Table 3, the TMN/6.7EO sample contains 16.7 wt % of unreacted TMN alcohol residue and has PDI of 2.59. The TMN/9.4EO sample contains 16.5 wt % unreacted TMN alcohol residue and has PDI of 2.43. The TMN/13.0EO sample contains 10.7 wt % unreacted TMN alcohol residue and has PDI of 2.09.

Following the same procedure, other TMN/EO products are prepared at listed in Table 3 (Example 13).

TABLE 3
Property Results for KOH Catalyzed TMN Ethoxylates and Alkoxylates

| | | | | | | | | | Unreacted |
|---|---|---|---|---|---|---|---|---|---|
| Ex/Sample | | | | Water^{a} | | | | | TMN |
| No. | Description | % OH | HEMW | (ppm) | Mₙ | M_{w} | M_{z} | PDI | (wt %) |
| 12-A | TMN/6.7 EO | 3.50 | 486 | 512 | 648 | 1,67 | 3,172 | 2.59 | 16.7 |
| 12-B | TMN/9.3 EO | 2.83 | 601 | — | 745 | 1,810 | 3,205 | 2.43 | 16.5 |
| 12-C | TMN/13.0 EO | 2.23 | 762 | — | 946 | 1,974 | 3,266 | 2.09 | 10.7 |
| 13-A | TMN/1.9 EO | 6.26 | 271 | 174 | 303 | 775 | 2,160 | 2.55 | 47.7 |
| 13-B | TMN/8.7 EO | 2.98 | 571 | — | 715 | 1,495 | 2,650 | 2.09 | 12.7 |

^{a}Water content in alcohol initiator

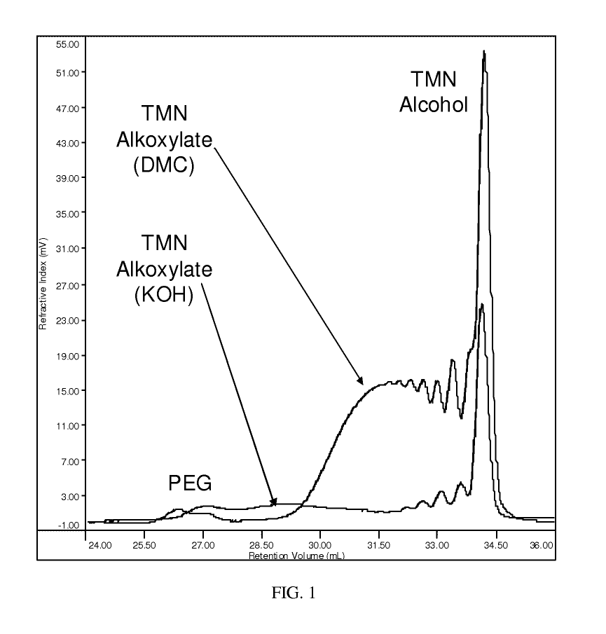

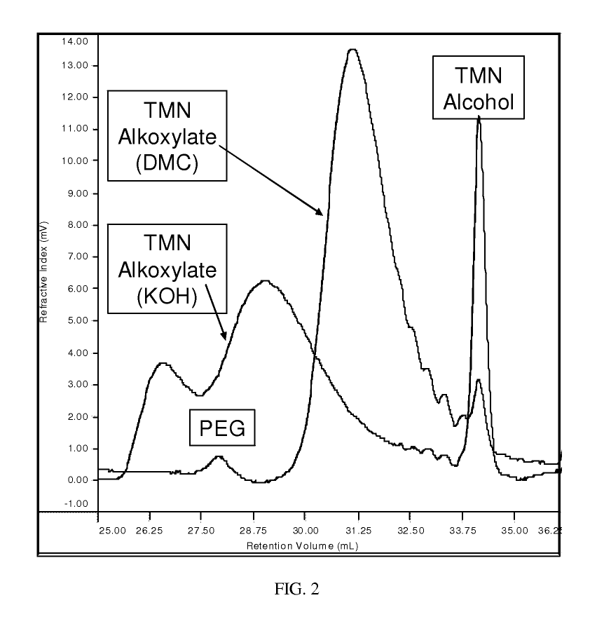

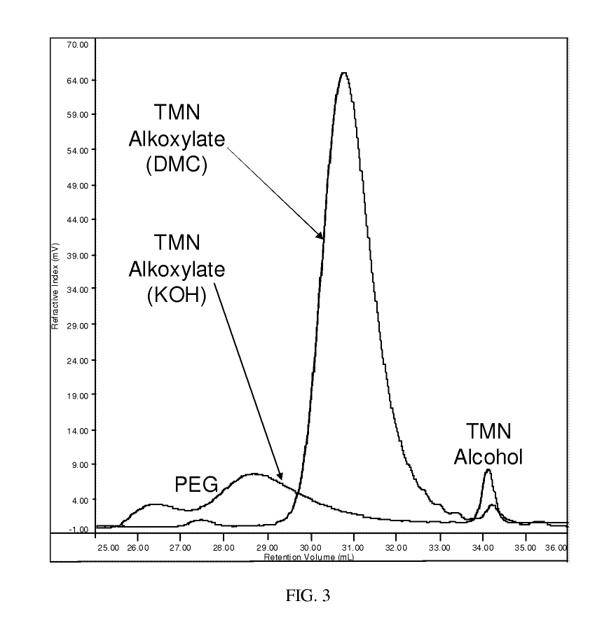

DMC catalyst converts more starting alcohol and has less impurities (polyethylene glycol, PEG) compared to KOH catalysis at similar EO/TMN charge ratios. FIGS. 1-3 compare the GPC chromatograms of alkoxylates made with DMC catalyst with alkoxylates made with KOH catalyst. FIG. 1 shows alkoxylation with 2.0 moles of EO, FIG. 2 shows alkoxylation with 6.6 mole of EO, and FIG. 3 shows alkoxylation with 9.5 mole of EO. PEG elutes at <29 minutes, the TMN alkoxylate product elutes between 29-33.6 minutes, and TMN alcohol elutes between 33.6-35.0 minutes.

### Examples 14

### Post Ethoxylation Processing

Even though the DMC catalyzed ethoxylation process of the invention results in product with desirably low polydispersity, in some cases it may also be desirable to further reduce the concentration of residual alcohol in the product, for instance in order to improve cloud point and/or to reduce odor. In general, the post ethoxylation processing involves heating the alkoxylate product under vacuum with a head space nitrogen purge and with agitation. Through post ethoxylation processing samples containing residual alcohol content of <1 wt % may be obtained. Exemplary data are shown in Table 4. The table shows the approximate temperature, pressure, and time used for the post processing. Residual alcohol content, cloud point and PDI of the final product are also shown.

TABLE 4
Post Processing of Various Ethoxylate Samples

| Product | | | | | | | |
|---|---|---|---|---|---|---|---|
| from | | | | | Cloud | | Residual |
| Ex/Sample | | Temp | Pressure | | Point | | TMN |
| No. | Description | (° C). | (Torr) | Time | (° C.) | PDI | (wt %) |
| 6-C | TMN/11.1 EO | 130 | 70 | 8 h | 76.7 | 1.05 | <0.03 |
| | | 175 | 200 | 1 h | | | |
| 7 | TMN/6.6 EO | 175 | 200 | 2.5 h | 36.5 | 1.10 | <0.03 |

While the invention has been described above according to its preferred embodiments, it can be modified within the spirit and scope of this disclosure. This application is therefore intended to cover any variations, uses, or adaptations of the invention using the general principles disclosed herein. Further, the application is intended to cover such departures from the present disclosure as come within the known or customary practice in the art to which this invention pertains and which fall within the limits of the following claims.

1. A composition comprising one or more alkoxylates of formula I: 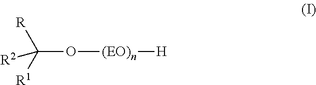
wherein EO is ethyleneoxy; n is 1-40; R and R¹ are independently C₁-C₁₄ alkyl; and R² is H or C₁-C₁₃ alkyl, wherein the group formed by R, R¹, R² and the carbon to which they are attached contains 7 to 16 carbon atoms and has a branching degree of at least 3, wherein the polydispersity index of the alkoxylates is 2.0 or less.

2. A composition according to claim 1 wherein the group formed by R, R¹, R² and the carbon to which they are attached contains 9 to 12 carbon atoms.

3. A composition according to claim 1 wherein the alkoxylate is of formula II: 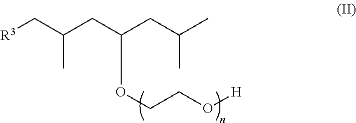
wherein R³ is H or iso-propyl and n is 3-40.

4. A composition according to claim 1 wherein the alkoxylate is of the formula: 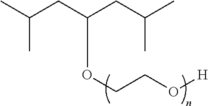
wherein n is 1-40.

5. A composition according to claim 1 wherein the alkoxylate is of the formula: 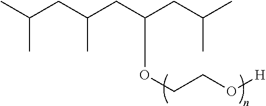
wherein n is 1-40.

6. A composition according to claim 1 wherein n ranges from about 8 to about 15.

7. A composition according to claim 1 wherein the polydispersity index is 1.15 or less.

8. A composition according to claim 1 comprising no more than 10 percent by weight of residual alcohol.

9. A process for making the alkoxylate composition of claim 1, comprising: reacting under alkoxylation conditions a secondary alcohol having 7 to 16 carbon atoms and a branching degree of 3 or more with ethylene oxide, wherein the alkoxylation is conducted in the presence of a double metal cyanide catalyst.

10. A process according to claim 9 wherein the secondary alcohol has 9 to 12 carbon atoms and a branching degree of 3 or more.

11. A process according to claim 9 wherein the secondary alcohol is 2,6,8-trimethyl-4-nonanol or 2,6-dimethyl heptan-4-ol.

12. A process according to claim 9 wherein the secondary alcohol is dried prior to the alkoxylation step in order to reduce residual water content to 200 ppm or less.

13. A process according to claim 9 wherein, following the alkoxylation reaction, the alkoxylate composition is heated at elevated temperature, under reduced pressure, or with an inert gas sparge, or any combination of the three, in order to remove at least a portion of residual alcohol contained in the composition.

14. A formulation selected from a detergent, hard surface cleaner, polyurethane formulation, epoxy formulation, emulsion polymerization formulation, thermoplastic formulation, metal product, agricultural product including herbicides and pesticides, oilfield product, pulp and paper product, textile formulation, water treatment product, flooring product, ink formulation, colorant formulation, pharmaceutical product, cleaning product, personal care product, fluororesin dispersion, and lubricant, wherein the formulation comprises a composition according to claim 1.
